(19) United States
(12) Patent Application Publication
(10) Pub. No.: US 2010/0068271 A1
(43) Pub. Date: Mar. 18, 2010

(54) Divisible Osmotic Dosage Forms and Methods of Use

(75) Inventors: Allan S. Kaplan, Boca Raton, FL (US); Lawrence Solomon, Miami, FL (US)

Correspondence Address: TED W. WHITLOCK REGISTERED PATENT ATTORNEY, P.A., 5323 SW 38TH AVENUE, FT. LAUDERDALE, FL, 33314, US

(73) Assignee: Accu-Break Technologies, Inc

(21) Appl. No.: 12/516,383

(22) PCT Filed: Nov. 29, 2007
(86) PCT No.: PCT/US07/85967

§ 371 Date: May 27, 2009

Related U.S. Application Data
(60) Provisional application No. 60/867,965, filed on Nov. 30, 2006.

(51) Int. Cl. A61K 9/28

(52) U.S. Cl. 424/474

(57) ABSTRACT

Described are specialized pharmaceutical dosage forms, e.g., osmotic delivery systems produced to be divisible into two useable half-strength tablets.

### FIELD OF THE INVENTION

The subject invention concerns specialized pharmaceutical dosage forms, e.g., osmotic delivery systems employed in tablets, that heretofore have not been disclosed to be divisible into two useable half-strength tablets.

### BACKGROUND

Certain layered dosage forms, such as tablets, have been developed that comprise an exit port provided in a semipermeable membrane, the membrane surrounding a composition comprising an active drug and an osmotic composition which imbibes aqueous fluids and swells to deliver the drug through the exit port. One embodiment of this osmotic drug delivery system provides a drug composition and the swellable osmotic composition as different, adjacent layers. The osmotic layer, formed from inactive excipients, is retained within the membrane and pushes out the drug layer as the osmotic layer swells and conforms to the space within the membrane. Such systems are described, for example, in U.S. Pat. Nos. 4,765,989, 4,783,337, 5,082,668, 5,200,194, and 5,795,591, each of which issued to Alza Corporation and are incorporated by reference herein in their entirety. These osmotic delivery systems are well known and are commonly referred to in the industry under the tradename, OROS®. Variations of the OROS delivery systems include providing more than one exit port, providing soluble plugs in the exit ports, and varying the compositions of the active and inactive layers for controlling the release rate of a drug. The delivery systems can be especially applicable for the delivery of low-solubility drugs.

It is specifically noted that these OROS systems, and dosage forms employing such systems, represent unit doses. Breaking these osmotic delivery tablets impairs the time-release characteristics of the technology and is specifically not recommended by the manufacturer or product marketer, as reflected by the package inserts for Procardia XL® and Glucotrol XL®.

It would represent an important and unexpected improvement in the layered or osmotic tablet art to provide tablets that incorporate the delivery characteristics described above and are also divisible, wherein the divided tablet parts (called “tablettes” herein) retain release kinetics for the drug or drugs contained within the tablet that are substantially the same. Such advantageous tablets, and methods for production and use of these tablets, are described herein.

### SUMMARY OF THE INVENTION

The invention involves pharmaceutical tablets comprising three or more layers and three or more segments. The terms “tablette,” “layer,” and “segment” are used in accordance with the definitions and descriptions provided in published PCT applications WO 05/112,870, WO 05/112,897, WO 05/112,898, WO 05/112,900, and WO 06/038,916, which are incorporated herein by reference in their entirety. The layers or segments are configured to provide a divisible osmotic dosage form that retains, in each portion of the broken dosage form, drug release characteristics substantially the same as the drug release characteristics for those compositions in the whole tablet, prior to breaking. Thus, drug release characteristics are not substantially altered by breaking of the whole tablet into tablet portions (tablettes) containing partial doses. These substantially equivalent drug release characteristics can be achieved by providing a divisible segment within the dosage form, positioned between two active segments in the intact dosage form. A semipermeable membrane can surround all or part of the whole dosage form and preferably surrounds at least the active segments.

In another embodiment, osmotic tablets can be produced, including a coating and exit port(s), and then one tablet could be adhesively joined (at the end opposite to the exit port) to one end of a preferably scored linker piece that may or may not contain active drug and may or may not comprise a plurality of layers. Another tablet could be adhesively joined (at the end opposite to the exit port) to the linker, such as at the opposite surface.

Typically, the drug amount and specific type of drug would be identical on the opposite ends of the tablet; however the structure or manufacturing method can be varied by the ordinarily skilled artisan to provide a different drug or combination of drugs in each respective active segment.

The invention involves methods of manufacture as are known in the art for multiple layer and coated tablets. Additionally, the methods can be varied, such as omitting the coating from a portion of the tablet, such as the breaking segment; or by providing a score in one or more of the segments, preferably the breaking segment (Layer 3 above) to assist in the optional tablet breaking. The scores can be formed during compression of the tablets or post-tabletting.

The invention further involves methods of breaking the tablet through the middle segment (which is usually, but not necessarily, also the middle layer). The invention further involves novel methods of treatment of patients with a fractional dose from a whole dose, in which the whole dose comprises a structure as described herein.

### BRIEF DESCRIPTION OF THE DRAWINGS

FIG. 1A shows a cross-section (through the longitudinal midline) of a three layer/three-segment coated, compressed tablet according to the subject invention.

FIG. 1B shows a variation of the dosage form of FIG. 1A, including a score.

FIG. 1C is a variation of the tablet of FIG. 1A, showing a coating that coats the middle layer/segment.

FIG. 1D shows an embodiment of the dosage form of FIG. 1C, where a score is formed into the membrane coating.

FIG. 1E shows a dosage form of FIG. 1A, broken through the middle layer/segment, providing two separate tablettes.

FIG. 2A shows a cross-section (through the longitudinal midline) of a five layer/five segment coated, compressed tablet according to the subject invention.

FIG. 2B shows a dosage form of FIG. 2A, broken through the middle layer/segment forming two separate tablettes.

FIG. 2C depicts a tablette from a dosage form of FIG. 2A or 2B, illustrating the influx of aqueous fluid (H₂O) and the egress of drug from the exit port.

FIGS. 3A-3D depict an embodiment of a dosage form according to the subject invention, as produced by adhering together two osmotic tablet portions. FIG. 3A shows a first tablet portion comprising a first and second layer/segment, and a semipermeable membrane coating the layers/segments.

FIG. 3B shows a tablet portion substantially the same as in FIG. 3A, but where the membrane entirely surrounds the first and second layers/segments.

FIG. 3C shows a linker segment to facilitate breaking of the dosage form into tablettes.

FIG. 3D shows a whole dosage form produced by adhering together the tablet portions and the linker shown in FIGS. 3A, 3B and 3C.

### DETAILED DESCRIPTION OF THE INVENTION

The invention enhances the usefulness of the known “push-pull” and related osmotic systems for controlled drug release, often zero-order release, by providing a readily divisible osmotic tablet that retains drug release characteristics as in the whole tablet, even after being divided. In one preferred embodiment, the invention involves two substantially identical bi-layer or tri-layer systems joined together by a divisible linker that is typically inactive. These systems can be prepared by forming each layer sequentially using a multiple layer tablet press, or by separately forming each bilayer or tri layer portion, and then adhering or otherwise adjoining each portion to the linker.

Thus, the invention can involve a bilayer system in which a first layer (segment) comprises an active drug or drugs, and a second layer (segment) typically comprises an inactive composition, but also may comprise a drug in the composition. In a preferred embodiment, two substantially identical layered drug delivery systems are adjoined at opposite ends of an inactive linker to form the whole dosage form. Thus, two bilayer systems are joined by a linker that preferably comprises an inactive, scored composition. The linker can be also be unscored, but preferably comprises a composition that can be readily broken manually or by using a bladed implement, such as a knife, razor, or commercially available tablet splitter device.

A typical system according to the subject invention comprises a semipermeable membrane as a coating on one or more segments of the dosage form. The segments of the tablet can be coated after compression. The purpose of the membrane is to allow fluids in, thus allowing the typically inactive layer to swell, creating an osmotic gradient against the active layer. One or more defects, holes, exit ports, or the like in the active layer allows egress of drug. The active layer itself can involve an immediate release composition or may itself be of a controlled release nature, such as in a matrix or microparticulate tablet formulation.

A typical five-layer (and five-segment) tablet of the invention can be prepared as follows:
Layer 1: Active composition (such as a granulation) comprising at least a half-therapeutic quantity of drug. The granulation may be immediate release or intrinsically controlled release, as with a matrix formulation.
Layer 2: Expanding “push” layer formed from swellable materials, preferably substantially drug free.
Layer 3: Composition, preferably substantially drug-free, providing a readily breakable segment. The composition may be substantially inert, such as an impermeable matrix, or may be swellable.
Layer 4: Identical to layer 2
Layer 5: Identical to layer 1.

A semi-permeable membrane is then applied to the tablet, or to certain segments of the tablet, either using known techniques, such as pan coating or spray coating, or by variants of such techniques that could involve grasping layer 3 and dipping the tablet in a coating solution.

All compositions of the above dosage form comprise pharmaceutically acceptable ingredients. Such pharmaceutically acceptable ingredients are described, for example, in Remington's Pharmaceutical Sciences 20th Ed., Mack Publishing Co., Easton, Pa. (2000), which is incorporated by reference.

One or more exit ports (holes, defects in the exterior of the tablet, etc.) would typically be created at opposite ends of the dosage form through the coating layer over layer 1 and layer 5, to allow drug to leave the tablet through said ports. Often, the exit ports are created with a laser. Typically the semipermeable membrane does not allow egress of any substantial quantity of drug through the membrane, though it does allow water to enter the tablet. Polymeric membranes used to regulate the entry of fluids such as water from the exterior to the interior of the system can be microporous as well as semipermeable.

The composition of layer 3 may optionally contain drug. In the most preferred embodiment, it Jacks drug. Layer 3 may optionally and preferably comprise a separation mark such as a score or printed indicia. These have been disclosed in the above-disclosed. PCT publication WO 05/112,870.

Other layers may be added to the tablet as desired and as technically feasible using a multilayer tablet press to produce an operable tablet.

Other types of osmotic tablets can also be made to provide a breakable segment, in accordance with the subject invention. For example, osmotic dosage forms similar to those described above for a five-layer/segment configuration, but wherein the drug from the active layer is dispensed by osmotically imbibing fluid from its aqueous environment and exiting through the port in the semipermeable membrane; however, these three-layer or three-segment dosage forms do not include a separate, swellable “push” composition as a separate layer or segment.

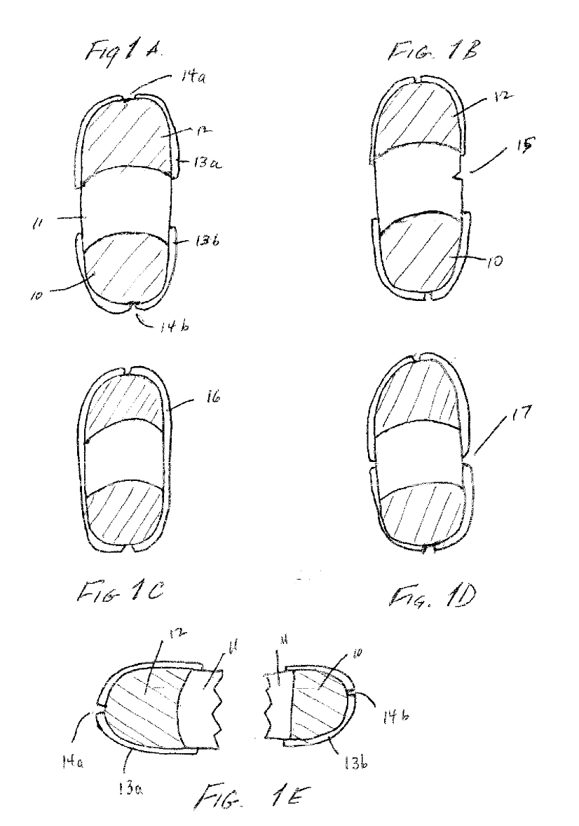

Exemplary configurations are shown in the attached drawings. FIG. 1A shows a cross-section (through the longitudinal midline) of a three layer/three-segment coated, compressed tablet comprising a first active layer/segment 10, preferably comprising an osmotic composition; a second inactive layer/segment 11, preferably comprising an inactive, substantially inert composition; and a third layer/segment 12, preferably substantially identical to the first layer/segment. The layered composition includes a semipermeable membrane (or membranes if non-contiguous) 13a and 13b coated onto the first and third layers/segments, and preferably contacting the second layer/segment. The membranes 13a and 13b also include exit ports 14a and 14b, respectively, formed at each end of the dosage form to allow egress of drug from the active layers when in a suitable environment.

FIG. 1B shows a variation of the dosage form of FIG. 1A, including a score 15 formed into the middle, second layer/segment.

FIG. 1C is a variation of the tablet of FIG. 1A, showing a coating 16 that further coats the middle, second layer/segment.

FIG. 1D shows an embodiment of the dosage form of FIG. 1c, where a score 17 is formed into the membrane coating 16.

FIG. 1E shows a dosage form of FIG. 1A, broken through the middle, second layer/segment into two separate tablettes. Preferably, the semipermeable membrane portions, 13a and 13b retain their integrity in relation to the layer compositions 10, 11, and 12, such that the osmotic function of the active compositions, following influx of aqueous fluid into the compartment containing the drug compositions, provides for drug egress, even when the dosage form is broken through the second segment.

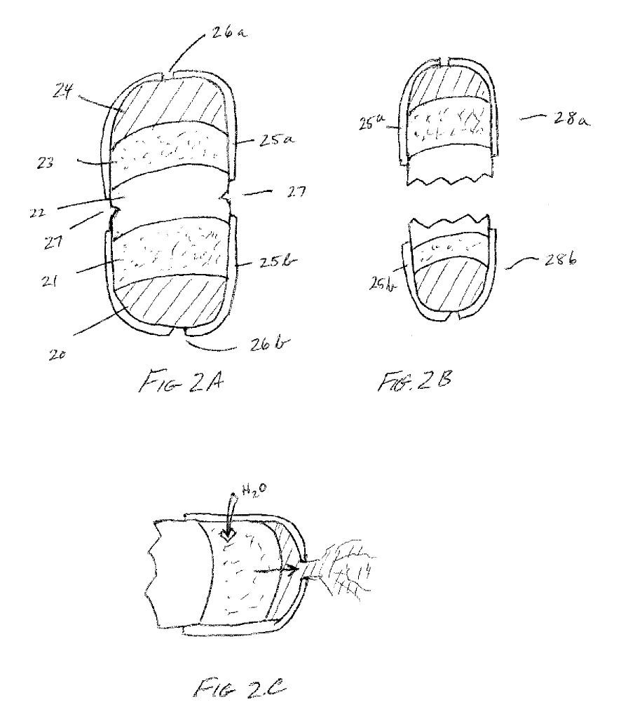

In FIGS. 2A-2C, a five layer/five segment dosage form is shown. FIG. 2A shows a cross-section (through the longitudinal midline) of a five layer/five segment coated, compressed tablet comprising a first active layer/segment 20; a second inactive layer/segment 21, preferably comprising an osmotic composition which is swellable or expands in an aqueous environment; a third layer/segment 22, preferably comprising an inactive, substantially inert composition; a fourth layer/segment 23, preferably substantially identical to the second layer/segment; and a fifth layer/segment 24, preferably substantially identical to the first layer/segment. The layered composition includes a semipermeable membrane (or membranes, if non-contiguous) 25a and 25b coated onto the first, second, fourth and fifth layers/segments, and preferably contacting the third layer/segment. The membranes 25a and 25b also include exit ports 26a and 26b, respectively, formed at each end of the dosage form to allow egress of drug from the active layers when in a suitable environment. This embodiment of the five layer/segment tablet also includes a score 27 formed into the middle, third layer/segment.

FIG. 2B shows a dosage form of FIG. 2A, broken through the middle, third layer/segment forming two separate tablettes 28a and 28b. Preferably, the semipermeable membrane portions, 25a and 25b retain their integrity in relation to the layer compositions 20-24 such that the osmotic function of the osmotic compositions, following influx of aqueous fluid into the dosage form, provides for drug egress form the active layers, even when the dosage form is broken through the second segment. The osmotic compositions absorb aqueous fluid from the environment, and thereby expand, forming a “push” layer to force active drug through the exit port of the dosage form. The influx of aqueous fluid (H₂O) and the egress of drug from the exit port are illustrated in FIG. 2C.

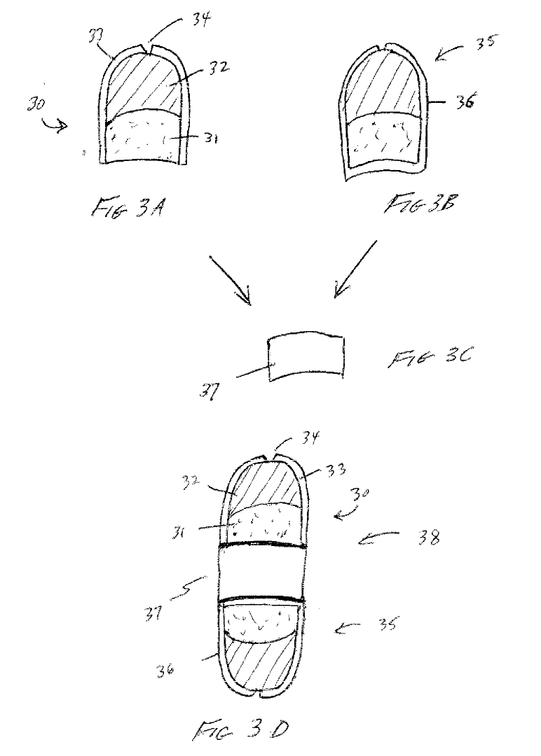

Another embodiment of the subject invention, namely a dosage form produced by adhering together two osmotic tablet portions, is illustrated in FIGS. 3A-3D. In FIG. 3A, a first tablet portion 30 comprising a first layer/segment 31, preferably an osmotic “push” composition, and a second layer/segment 32, preferably comprising an active drug composition, is provided, having a semipermeable membrane coating the layers/segments. The membrane coating includes an exit port 34 formed therein. In this embodiment shown, the membrane does not entirely surround the first layer/segment 31. A second tablet portion, identical to the tablet portion 30, or a tablet portion 35 as shown in FIG. 3B can be provided. The tablet portion 35 is substantially identical to tablet portion 30, except that membrane coating 36 surrounds the entire tablet portion 35. In addition, a linker segment 37, preferably comprising a substantially inert composition that can be readily broken through, or provided with a score to facilitate breaking through the segment, as shown in FIG. 3C, can be provided in order to adjoin tablet portions (e.g., 30 and 35) together to form a single dosage form 38 (FIG. 3D) that can be readily divisible through segment 37.

In the manufacture of dosage forms according to the subject invention, a breakable tablet can be formed on a suitable tablet press, such as the Korsch TRP900 multiple layer tablet press. The manufacturing process can be completed in three layers resulting in three segments or in five layers resulting in at least three, and typically five, segments. The first (bottom) active segment can be formed from one or more layers. The second impermeable matrix/possibly swellable segment can be formed from one or more layers (which may form one or more segments). The third (top) active osmotic segment can be formed from one or more layers. The tablets are then coated in a suitable coating pan according to the methods commonly known to those skilled in the art of tablet coating with a suitable semipermeable membrane. An appropriate sized hole is then made in the active ends of the tablet using a laser or some appropriate mechanical means as described above. Subsequently the tablets are optionally printed and/or scored as described above for the “push/pull” type “OROS” tablets.

In another embodiment of the invention, a bilayer system as described above could be linked to a different drug layer or segment, to a different push-pull system such as one comprising a different drug or drugs, or different release pattern of the same drug or drugs, or to an immediate release composition or compositions.

Many drugs are suitable for the bilayer or trilayer system of the invention, such as nifedipine, amlodipine, other dihydropyridines, glipizide and other sulfonylureas, methylphenidate, and other biguanides. No limitation on the types of drug or drugs present in any part of the dosage form is implied by any statements herein.

Several manufacturing methods may be employed in the invention. These include the following.

1. Create a bilayer (two-segment) or trilayer (two- or three-segment) structure in a tablet press, eject from the press, coat the system with a semipermeable membrane or other suitable coating to accomplish the controlled release function. Create with a laser, a mechanical instrument, or other suitable method, a hole or holes in the first layer or segment, as opposed to the swellable and usually inactive layer or segment.

Take two of the above structures, each of which could be a dosage form, and using a suitable biologically safe adhesive substance such as shellac or an Eudragit or hydroxypropyl methylcellulose (HPMC), join the two structures at the end opposite to the hole to a third structure which is positioned in between the two tablets. This third linking structure is unlimited in such matters as size, shape, color, presence or absence of drug, solubility in gastric or intestinal fluid, and the like. In many preferred embodiments, it will be indented or marked to aid elective splitting.

Typically the two tablets that are linked together to form the final dosage form are in this embodiment identical in all material respects, such as in drug and dosage of said drug.

2. In a different preferred embodiment, one of the above structures is adhesively joined to a different structure, which may be a conventional monolayer compressed tablet, a different push-pull type system, or other structure containing a drug.

3, in a tablet press, dosage forms of the invention may be created using a five-layer press such as the TRP 900 manufactured by Korsch AG of Germany. A seven-layer press is technically feasible and when created could be used as well.

In a preferred five-layer, five-segment embodiment, a composition that forms the bottom layer of the dosage form enters the die. It contains a drug or drugs and is not a swellable layer. This layer may be tamped or lightly compressed according to standard techniques. A second layer comprising a swellable composition enters the die on top of said first layer. Tamping again may occur. A third layer of variable composition enters the die. In the most preferred embodiments, it lacks a drug. It may be an impermeable substance. It may have swellable characteristics. A fourth layer then enters the die on top of said third layer. It may be identical to the second layer. Tamping may occur of any of these layers. A fifth, uppermost end layer that may be identical to said first, bottom end layer then enters the die and final compression occurs.

Following tablet formation, tablet coating occurs, or at least coating of the first and second and fourth and fifth layers (now called the second and fourth segments), and preferably all layers (segments) except, optionally, the fourth. Care is taken during the coating process to keep the tablet intact. One method of coating the tablet is to grasp the third layer (segment) with a tool and dip the tablet into the coating medium that may be a solution, keeping the tablet in said medium for the appropriate length of time.

Characteristics of the linking middle (third, in a five-layer dosage form of the invention are not limited. An important consideration is the swellable second and fourth layers. Osmotic forces may cause the push feature to weaken as more of the swelling moves in the direction away from the first (or fifth) adjacent layer (segment). Appropriate consideration of this phenomenon is required and may necessitate that the middle segment have just enough swelling capability to provide secure contact between the middle layer and the semipermeable membrane coating the tablet to prevent movement of the middle layer without meaningfully affecting drug release.

Optionally and preferably, a score is created after dosage form creation into the linking segment (layer or possibly layers). This may be accomplished with a knife or hand-held instrument. An apparatus as has been disclosed by Kaplan and Solomon to score and optionally print a tablet or other dosage form post compression, such as in a transverse manner, may be used in appropriate fashion to create the score. It may be necessary if a tough or resilient semipermeable membrane surrounds the entire dosage form to abrade the membrane in the area of the score.

Hole or exit port creation in the top and bottom (e.g., the first and fifth layers in the above examples) has been typically performed with a laser. Other mechanical, chemical, or other techniques may also be utilized. No limitation in the number of holes per segment is intended.

A preferred method of use of the dosage form of the invention is for the intended user to ingest it whole. Another preferred use is to break through the linker segment and then ingest either tablette (broken tablet part) thereby formed, as desired.

Any suitable drug or drugs may be used in the dosage forms or layers of the invention. Lists of suitable drugs are available in numerous places. The PCT filings of Solomon and Kaplan list several. The Physicians' Desk Reference, 2006, lists numerous suitable drugs or drug combinations.

The dosage forms or tablet subunits or layers of the invention may themselves be layered externally with drug or a composition containing a drug.

It will be evident to ones skilled in the pharmaceutical and medical arts that the improvements and embodiments described herein cannot encompass every possibility.

1. A dosage form comprising a pharmaceutical tablet comprising:
a. top and bottom layers comprising a drug or drugs in a pharmaceutically effective quantity, wherein said layers comprise a coating having at least one exit port for the release of said drug or drugs therethrough;
b. one or more middle layers between the top and bottom layers comprising a swellable substance or substances; and
c. where there are two or more swellable middle layers, an optional breaking layer comprising an inert or inactive composition different than the composition of a middle layer, said breaking layer located between the two or more middle layers.

2. The dosage form as in claim 1 comprising a pharmaceutical tablet comprising:
a. a coated first layer containing drug and optionally comprising an exit port or ports for said drug, said layer representing a bottom layer;
b. a second layer disposed upon said first layer, comprising a swellable pharmaceutically acceptable composition;
c. a third layer disposed upon a swellable layer, said third layer comprising an inert or inactive pharmaceutically acceptable composition separation mark such as a score;
d. a fourth layer disposed above said third layer, said fourth layer comprising a swellable pharmaceutically acceptable composition; and
e. a coated fifth layer containing drug and optionally comprising an exit port or ports for said drug, said layer representing a top layer.

3. The dosage form of claim 1 consisting essentially of:
a. a coated first, bottom segment comprising one or more layers containing at least about a half-therapeutic amount of a drug;
b. a second segment comprising at least one swellable substance, disposed above and contiguous with said first, bottom segment;
c. a third segment disposed above and contiguous with said second segment;
d. a fourth segment disposed above and contiguous with said third segment, said fourth segment comprising a swellable substance; and
e. a coated fifth, top segment disposed above and contiguous with said fourth segment, comprising a drug in at least a half-therapeutic quantity, said coated segment comprising at least one exit port for said drug.

4. The dosage form of claim 1 in which the coating forms a semipermeable membrane around all or part of the dosage form.

5. The dosage form as in claim 1 in which the layer located between the middle layers is substantially free of drug.

6. The dosage form as in claim 1 in which the layer located between the middle layers is a substantially impermeable composition.

7. A method of obtaining a partial dose of a drug or drugs in an intact active layer by
a. providing a tablet of claim 1;
b. breaking through an inactive layer of said tablet to provide an intact active layer and a broken or divided portion of the inactive layer, wherein the partial dose has substantially the same release profile as exhibited from that layer in a whole or intact dosage form.

8. The method of claim 7 in which the partial dose is a half dose.

9. A tablette comprising an intact active segment and a broken or divided portion of an inactive segment formed by breaking through the inactive segment of a tablet of claim 1.

10. The tablette of claim 9 wherein the active segment provides substantially the same drug release profile after separation from the whole dosage form as exhibited for that segment in an unbroken or undivided whole or intact tablet.
